(19) United States
(12) Patent Application Publication
(10) Pub. No.: US 2024/0415563 A1
(43) Pub. Date: Dec. 19, 2024

(54) CHEMICAL NEUROMODULATION AGENT DELIVERY

(71) Applicant: Medtronic Ireland Manufacturing Unlimited Company, Dublin (IE)

(72) Inventors: Gerry Oliver McCaffrey, Abbeyknockmoy (IE); Cian Walsh, Oranmore (IE); Kelsey Sandquist, Cotati, CA (US); Enda Carter, Galway (IE); Gerry Broderick, Oranmore (IE)

(21) Appl. No.: 18/699,551

(22) PCT Filed: Oct. 25, 2022
(86) PCT No.: PCT/EP2022/079793

§ 371 Date: Apr. 8, 2024

Related U.S. Application Data
(60) Provisional application No. 63/273,725, filed on Oct. 29, 2021.

(51) Int. Cl. A61B 18/06; A61B 18/00; A61B 18/04; A61M 25/00; A61M 25/10

(52) U.S. Cl. CPC A61B 18/06; A61M 25/0084; A61M 25/1002; A61B 2018/0022; A61B 2018/00434; A61B 2018/00511; A61B 2018/00577; A61B 2018/046; A61M 2025/0089; A61M 2025/1047; A61M 2025/1072

(57) ABSTRACT

An example catheter includes an elongated member configured to be navigated through vasculature of a patient to a target treatment site in a vessel of the patient, a needle positioned at a distal portion of the elongated member, the needle configured to be within the elongated member when in a delivery configuration and radially extended to puncture a vessel wall of the vessel at the target treatment site when in a deployed configuration, and an expandable balloon including a needle guide configured to position a distal end of the needle at a predetermined distance from a central axis of the elongated member when the balloon is in an expanded configuration and the needle is in the deployed configuration.

### TECHNICAL FIELD

The present technology is related to chemical neuromodulation.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

### SUMMARY

The present technology is directed to devices, systems, and methods for neuromodulation, such as renal neuromodulation. The devices, systems, and methods disclosed herein improve the accuracy and/or precision of axial/longitudinal positioning, circumferential positioning, and/or penetration depth of one or more renal neuromodulation needles. Although renal neuromodulation is primarily described herein, devices, systems, and techniques described herein may be applied to other types of neuromodulation, such as neuromodulation performed on nerves other than the renal nerves, at sites other than within a renal vessel, or both. In general, the devices, systems, and techniques described herein may be used to perform neuromodulation from within any suitable anatomical lumen that has nerves adjacent to the anatomical lumen.

In one example, this disclosure describes a catheter including an elongated member configured to be navigated through vasculature of a patient to a target treatment site in a vessel of the patient; a needle positioned at a distal portion of the elongated member, the needle configured to be within the elongated member when in a delivery configuration and radially extended to puncture a vessel wall of the vessel at the target treatment site when in a deployed configuration; and an expandable balloon including a needle guide configured to position a distal end of the needle at a predetermined distance from a central axis of the elongated member when the balloon is in an expanded configuration and the needle is in the deployed configuration.

In another example, this disclosure describes a method including navigating a catheter through vasculature of a patient to a target treatment site in a vessel of the patient, wherein the catheter comprises: an elongated member configured to be navigated through vasculature of a patient to the target treatment site; a needle positioned at a distal portion of the elongated member, the needle configured to be within the elongated member when in a delivery configuration and radially extended to puncture a vessel wall of the vessel at the target treatment site when in a deployed configuration; and an expandable balloon including a needle guide configured to position a distal end of the needle at a predetermined distance from a central axis of the elongated member when the balloon is in an expanded configuration and the needle is in the deployed configuration; and expanding the balloon within the vasculature to maintain the elongated member substantially stationary relative to the vessel wall.

In another example, this disclosure describes a chemical ablation system including: an ablation catheter including an elongated member configured to be navigated through vasculature of a patient to a target treatment site in a vessel of the patient; a needle positioned at a distal portion of the elongated member, the needle configured to be within the elongated member when in a delivery configuration and radially extended to puncture a vessel wall of the vessel at the target treatment site when in a deployed configuration; and an expandable balloon including a needle guide configured to position a distal end of the needle at a predetermined distance from a central axis of the elongated member when the balloon is in an expanded configuration and the needle is in the deployed configuration; and a medical device communicatively coupled to a lumen of the needle and configured to control delivery of a fluid via the lumen to the target treatment site.

In another example, this disclosure describes a catheter including an elongated member configured to be navigated through vasculature of a patient to a target treatment site in a vessel of the patient; a needle positioned at a distal portion of the elongated member, the needle configured to be within the elongated member when in a delivery configuration and radially extended to puncture a vessel wall of the vessel at the target treatment site when in a deployed configuration; and an expandable inverted neck balloon including: when in an expanded configuration, a perimeter longitudinal length at a radial perimeter of the inverted neck balloon that is greater than an inner longitudinal length at a radially inwards position of the inverted neck balloon proximate the elongated member, wherein the expandable inverted neck balloon is configured to position a distal end of the needle at a predetermined distance from a central axis of the elongated member when the balloon is in the expanded configuration and the needle is in the deployed configuration.

In another example, this disclosure describes a catheter including an elongated member configured to be navigated through vasculature of a patient to a target treatment site in a vessel of the patient; a needle positioned at a distal portion of the elongated member, the needle configured to be within the elongated member when in a delivery configuration and radially extended to puncture a vessel wall of the vessel at the target treatment site when in a deployed configuration; and an expandable balloon configured to position a distal end of the needle at a predetermined distance from a central axis of the elongated member when the balloon is in an expanded configuration and the needle is in the deployed configuration, wherein, when the balloon is in the expanded configuration, the balloon is configured to apply a force to the vessel wall to urge a portion of the vessel contacted by the balloon to straighten.

Also disclosed herein is a catheter that includes an elongated member configured to be navigated through vasculature of a patient to a target treatment site in a vessel of the patient, a needle positioned at a distal portion of the elongated member, the needle configured to be within the elongated member when in a delivery configuration and radially extended to puncture a vessel wall of the vessel at the target treatment site when in a deployed configuration, and an expandable balloon including a needle guide configured to position a distal end of the needle at a predetermined distance from a central axis of the elongated member when the balloon is in an expanded configuration and the needle is in the deployed configuration.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout.

FIG. 1 is a partially schematic illustration of a neuromodulation system configured in accordance with some examples of the present disclosure.

FIG. 2 illustrates a technique for accessing a renal artery and modulating renal nerves with the system of FIG. 1 in accordance with some examples of the present disclosure.

FIG. 3 is a conceptual illustration of an example sympathetic nervous system (SNS) and how the brain communicates with the body via the SNS.

FIG. 4 is an enlarged anatomic view of nerves innervating a left kidney to form the renal plexus surrounding the left renal artery.

FIGS. 5 and 6 are anatomic and conceptual views, respectively, of a human body depicting neural efferent and afferent communication between the brain and kidneys.

FIGS. 7 and 8 are anatomic views of the arterial vasculature and venous vasculature, respectively, of a human.

FIG. 9A is a conceptual cross-sectional side view of an example neuromodulation catheter that includes a distal portion of an elongated shaft in a delivery configuration and positioned within a renal vessel, in accordance with some examples of the present disclosure.

FIG. 9B is a conceptual cross-sectional axial view of the example neuromodulation catheter of FIG. 9A, in accordance with some examples of the present disclosure.

FIG. 10A is a conceptual cross-sectional side view of the example neuromodulation catheter of FIG. 9A in a partially deployed configuration and positioned within renal vessel, in accordance with some examples of the present disclosure.

FIG. 10B is a conceptual cross-sectional axial view of the example neuromodulation catheter of FIG. 10A, in accordance with some examples of the present disclosure.

FIG. 11A is a conceptual cross-sectional side view of the example neuromodulation catheter of FIG. 9A in a deployed configuration and positioned within a renal vessel, in accordance with some examples of the present disclosure.

FIG. 11B is a conceptual cross-sectional axial view of the example neuromodulation catheter of FIG. 11A, in accordance with some examples of the present disclosure.

FIG. 12 is a conceptual cross-sectional side view of another example neuromodulation catheter that includes a distal portion of an elongated shaft of in a delivery configuration and positioned within a main renal artery, in accordance with some examples of the present disclosure.

FIG. 13 is a conceptual cross-sectional axial view of the example neuromodulation catheter of FIG. 12, in accordance with some examples of the present disclosure.

FIG. 14 is a conceptual cross-sectional side view of the example neuromodulation catheter of FIG. 12 in a partially deployed configuration and positioned within a main renal artery, in accordance with some examples of the present disclosure.

FIG. 15 is a conceptual cross-sectional axial view of the example neuromodulation catheter of FIG. 14 at a first longitudinal position, in accordance with some examples of the present disclosure.

FIG. 16 is a conceptual cross-sectional axial view of the example neuromodulation catheter of FIG. 14 at a second longitudinal position, in accordance with some examples of the present disclosure.

FIG. 17 is a conceptual cross-sectional side view of another example neuromodulation catheter that includes a distal portion of an elongated shaft of in a deployed configuration and positioned within a main renal artery, in accordance with some examples of the present disclosure.

FIG. 18 is a perspective view of the example neuromodulation catheter of FIG. 17 in a partially deployed configuration, in accordance with some examples of the present disclosure.

FIG. 19 is a conceptual cross-sectional side view of the example neuromodulation catheter of FIG. 17 at a first stage of attaching an expandable stabilizer, in accordance with some examples of the present disclosure.

FIG. 20 is a conceptual cross-sectional side view of the example neuromodulation catheter of FIG. 17 at a second stage of attaching an expandable stabilizer, in accordance with some examples of the present disclosure.

FIG. 21 is a conceptual cross-sectional side view of the example neuromodulation catheter of FIG. 17 at a third stage of attaching an expandable stabilizer, in accordance with some examples of the present disclosure.

FIG. 22 is a conceptual perspective view of an example neuromodulation catheter illustrating a notch-and-groove mechanical guidance feature at a distal portion of the example neuromodulation catheter, in accordance with some examples of the present disclosure.

FIG. 23 is a flow diagram illustrating an example technique of performing neuromodulation of renal nerves in accordance with some examples of this disclosure.

FIG. 24 is a flow diagram illustrating another example technique of performing neuromodulation of renal nerves in accordance with some examples of this disclosure.

### DETAILED DESCRIPTION

The present technology is directed to devices, systems, and methods for neuromodulation, such as renal neuromodulation, using chemical agents. Although the following examples will be described primarily with respect to renal neuromodulation, a person having ordinary skill in the art will understand that the devices, systems, and methods described herein may be used for neuromodulation at any suitable intravascular location within a body of a patient.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Conditions such as arrhythmias, hypertension, states of volume overload (e.g., heart failure), and progressive renal disease due to excessive activation of the renal sympathetic nervous system (SNS), may be mitigated by modulating the activity of overactive nerves (neuromodulating), for example, denervating or reducing the activity of the overactive nerves. Sympathetic nerves of the kidneys terminate in the renal blood vessels, the juxtaglomerular apparatus, and the renal tubules, among other structures. The overactive nerves may be chemically or electrically denervated by ablating sympathetic nerve tissue in or near renal blood vessels. A chemical or electrical energy may be delivered to the sympathetic tissue via navigating a catheter including needles and/or electrodes within the vasculature of the patient. In the case of chemical renal denervation, one or more needles may radially extend from the catheter to puncture a vessel wall to deliver the chemical and/or a cold therapy fluid via a needle lumen to ablate tissue at a target treatment site.

Renal neuromodulation, such as renal denervation, may be accomplished using one or more of a variety of treatment modalities, including radio frequency (RF) energy, microwave energy, ultrasound energy, a chemical agent, or the like. When using a chemical agent, a neuromodulation catheter may be delivered to a renal vessel, such as a renal artery, of a patient. The neuromodulation catheter may include at least one port or needle through which the chemical agent is delivered. The chemical agent may be selected to modulate activity of one or more renal nerves adjacent to the renal artery in which the neuromodulation catheter is positioned. For example, the chemical agent may be a neurotoxic chemical selected to chemically ablate the one or more renal nerves near the renal artery.

In some examples, renal neuromodulation may require a relatively high degree of accuracy and precision in positioning needles when puncturing a vessel wall to deliver a chemical agent adjacent to one or more renal nerves. For example, a clinician may individually position needles via a neuromodulation catheter in an iterative fashion while receiving positioning feedback via an imaging system to ensure delivery of the chemical agent to the target tissue. The procedure may be done “by hand” and depend on the dexterity, patience, and stamina of the clinician, and may further be time-consuming.

In accordance with techniques of this disclosure, a neuromodulation catheter may include one or more features configured to position and/or stabilize therapeutic elements, e.g., configured to deliver the chemical agent, to improve the precision and accuracy of deployment of the therapeutic elements and delivery of the chemical agent to target tissue. In some examples, a neuromodulation catheter includes an expandable stabilizer, such as an expandable balloon, expandable wire basket, and the like, including a needle guide configured to guide a needle, when deployed, to position a distal end of the needle at a predetermined distance from a central axis of the catheter when the expandable stabilizer is expanded.

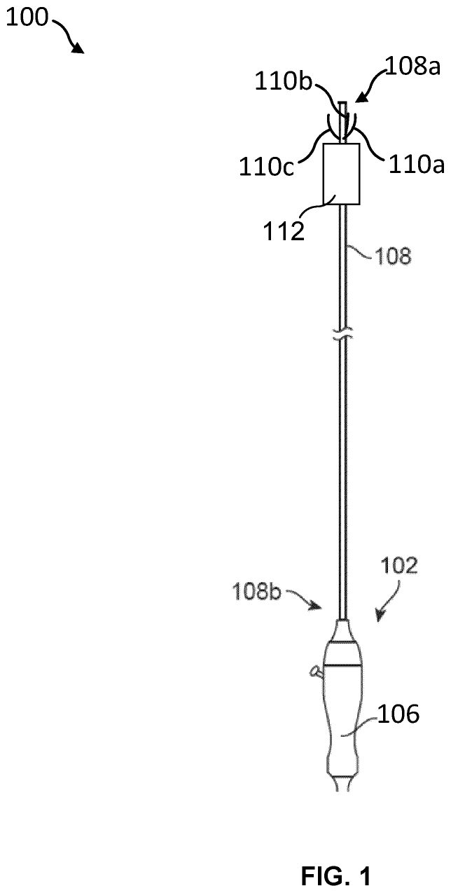

FIG. 1 is a partially schematic illustration of a neuromodulation system 100 (“system 100”) configured in accordance with some examples of the present technology. As shown in FIG. 1, system 100 includes a neuromodulation catheter 102. Neuromodulation catheter 102 includes a handle 106 and an elongated member, e.g., elongated shaft 108 attached to the handle 106. The elongated shaft 108 may include a distal portion 108a and a proximal portion 108b. Distal portion 108a of neuromodulation catheter 102 includes a plurality of therapeutic elements 110a-110c (“therapeutic elements 110”) and at least one expandable stabilizer 112. Therapeutic elements 110 and expandable stabilizer 112 may be positioned around (e.g., distributed around) a circumference of distal portion 108a.

Although distal portion 108a is shown in FIG. 1 as including three therapeutic elements 110 positioned around a circumference of distal portion 108a at a single longitudinal position along elongated shaft 108, in other examples, distal portion 108a may include any number of therapeutic elements 110. For example, distal portion 108a may include two, three, four, or more therapeutic elements 110 positioned around a circumference of distal portion 108a at a single longitudinal position. As another example, distal portion 108a may include positioned two, three, four, or more therapeutic elements 110 positioned around a circumference of distal portion 108a at each of multiple longitudinal positions along distal portion 108a. In examples in which distal portion 108a includes therapeutic elements 110 positioned at different longitudinal positions, each longitudinal position may include one or more therapeutic element 110, and each longitudinal position may include the same number of therapeutic elements 110, or one longitudinal position may include a different number of therapeutic elements 110 than one or more other longitudinal positions.

Although distal portion 108a is shown in FIG. 1 as including one expandable stabilizer 112 positioned around the circumference of distal portion 108a at a single longitudinal position along elongated shaft 108 proximal to therapeutic elements 110, in other examples, distal portion 108a may include any number of expandable stabilizers 112. For example, distal portion 108a may include two, three, four, or more expandable stabilizers 112 positioned around a circumference of distal portion 108a at a single longitudinal position. As another example, distal portion 108a may include one, two, three, four, or more expandable stabilizers 112 positioned around a circumference of distal portion 108a at each of multiple longitudinal positions along distal portion 108a and positioned distal or proximal to therapeutic elements 110. In examples in which distal portion 108a includes expandable stabilizers 112 positioned at different longitudinal positions, each longitudinal position may include one or more expandable stabilizer 112, and each longitudinal position may include the same number of expandable stabilizers 112, or one longitudinal position may include a different number of expandable stabilizers 112 than one or more other longitudinal positions. In some examples, expandable stabilizer 112 may include an inflatable balloon, an expandable wire basket, and the like.

Elongated shaft 108 may have any suitable outer diameter, and the diameter can be constant along the length of elongated shaft 108 or may vary along the length of elongated shaft 108. In some examples, elongated shaft 108 can be 2, 3, 4, 5, 6, or 7 French or another suitable size.

Distal portion 108a of elongated shaft 108 is configured to be moved within an anatomical lumen of a human patient to locate therapeutic elements 110 at a target site within or otherwise proximate to the anatomical lumen. For example, elongated shaft 108 may be configured to position therapeutic elements 110 within a blood vessel, a ureter, a duct, an airway, or another naturally occurring lumen within the human body. The following description focuses on positioning distal portion 108a and therapeutic elements 110 within a blood vessel. A person having ordinary skill in the art will understand that the description and examples described herein are also applicable to positioning distal portion 108a and therapeutic elements 110 within other anatomical lumens. In certain examples, intravascular delivery of the therapeutic elements 110 includes percutaneously inserting a guidewire (not shown) into a blood vessel of a patient and moving elongated shaft 108 and/or therapeutic elements 110 along the guidewire until therapeutic elements 110 reaches a target site (e.g., a renal vessel, such as a renal artery or renal vein). For example, the distal end of elongated shaft 108 may define a passageway for engaging the guidewire for delivery of therapeutic elements 110 using over-the-wire (OTW) or rapid exchange (RX) techniques. In other examples, neuromodulation catheter 102 can be a steerable or non-steerable device configured for use without a guidewire. In still other examples, neuromodulation catheter 102 can be configured for delivery via a guide catheter or sheath (not shown), or other guide device.

Once at the target site, therapeutic elements 110 can be configured to deliver therapy, such as RF energy, microwave energy, ultrasound energy, a chemical agent, or the like to provide or facilitate neuromodulation therapy at the target site. For ease of description, the following discussion will be primarily focused on delivering a chemical agent, such as a neurotoxic chemical, in which examples therapeutic elements 110 include needles. It will be understood, however, that therapeutic elements 110 may include elements or structures configured to deliver other types of therapy. For example, therapeutic elements 110 may include needle electrodes configured to deliver RF energy for RF ablation of nerves near the blood vessel in which neuromodulation catheter 102 is positioned.

In examples in which neuromodulation catheter 102 is configured to deliver a chemical agent, therapeutic elements 110 may include needles configured to be deployed to extend radially from distal portion 108a and at least partially pierce and/or puncture a wall of the blood vessel in which distal portion 108a is positioned. The needles may extend to and/or through the intima, media, and/or adventitia of the wall and be configured to deliver the chemical agent to the adventitia and/or peri-adventitia, in which renal nerves are located. By having therapeutic elements 110 located around a circumference of distal portion 108a, neuromodulation catheter 102 may be used to deliver the chemical agent around a circumference of the blood vessel in which distal portion 108a is positioned. While a circumference of the blood vessel is generally referred to herein, the blood vessel may not be perfectly circular in cross-section and may have any suitable geometry in cross-section.

Once at the target site, expandable stabilizer 112 can be configured to stabilize and/or maintain at least a portion of distal portion 108a substantially stationary relative to the wall of the blood vessel in which distal portion 108a is positioned. Expandable stabilizer 112 may also be configured to position at least a portion of distal portion 108a within the blood vessel, e.g., to radially center distal portion 108a within the blood vessel. Expandable stabilizer 112 may also include one or more guides configured to guide one or more of therapeutic elements 110 to a deployed configuration, e.g., to guide therapeutic elements 110 when radially extending from distal portion 108a to at least partially pierce and/or puncture the wall of the blood vessel. In some examples, expandable stabilizer 112 may be configured to position a distal end of a needle of therapeutic elements 110 to be at a predetermined distance from a central axis of elongated shaft 108, or distal portion 108a, when the therapeutic elements 110 and/or needle is in the deployed configuration, e.g., radially extended. In this way, expandable stabilizer may facilitate accurate and/or precise delivery of the chemical agent to target tissue.

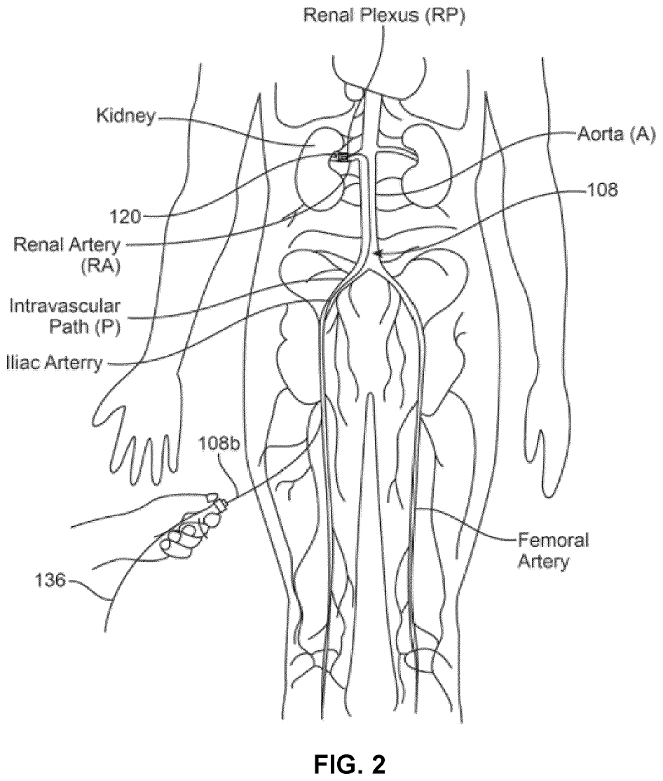

FIG. 2 (with additional reference to FIG. 1) illustrates gaining access to renal nerves of an example patient in accordance with some examples of the present technology. Neuromodulation catheter 102 provides access to the renal plexus RP through an intravascular path P, such as a percutaneous access site in the femoral (illustrated), brachial, radial, or axillary artery to a targeted treatment site within a respective renal artery RA. By manipulating proximal portion 108b of elongated shaft 108 from outside the intravascular path P, a clinician may advance at least distal portion 108a of elongated shaft 108 through the sometimes tortuous intravascular path P and remotely manipulate distal portion 108a (FIG. 1) of elongated shaft 108.

In the example illustrated in FIG. 2, therapeutic elements 110 are delivered intravascularly to the treatment site using a guidewire 136 in an OTW technique. A neuromodulation assembly 120 may define a passageway for receiving guidewire 136 for delivery of the neuromodulation catheter 102 using either an OTW or a RX technique. At the treatment site, guidewire 136 can be at least partially withdrawn or removed, and therapeutic elements 110 can transform or otherwise be moved to a deployed arrangement for delivering a chemical agent. In other examples, therapeutic elements 110 may be delivered to the treatment site within a different guide device, such as a guide sheath (not shown), with or without using guidewire 136. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the treatment site without the aid of guidewire 136 and/or a guide sheath.

An imaging device may enable image guidance, e.g., computed tomography (CT), fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal portion 108a and therapeutic elements 110. For example, a fluoroscopy system (e.g., including a flat-panel detector, x-ray, or c-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic elements 110. Further, in some examples, image guidance components (e.g., IVUS, OCT) may be integrated with neuromodulation catheter 102 and/or run in parallel with neuromodulation catheter 102 to provide image guidance during positioning of therapeutic elements 110. For example, image guidance components (e.g., IVUS or OCT) can be coupled to therapeutic elements 110 to provide three-dimensional images of the vasculature proximate the target site to facilitate positioning or deploying therapeutic elements 110 within the target renal blood vessel.

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally-induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery and relative to other physiological structures (such as an accessory renal artery), delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

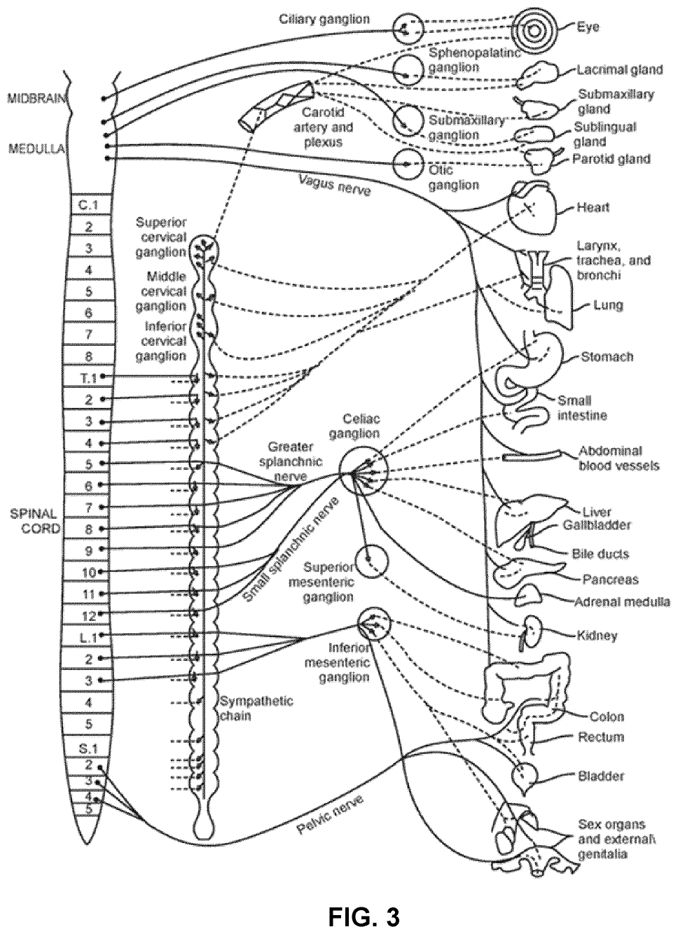

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

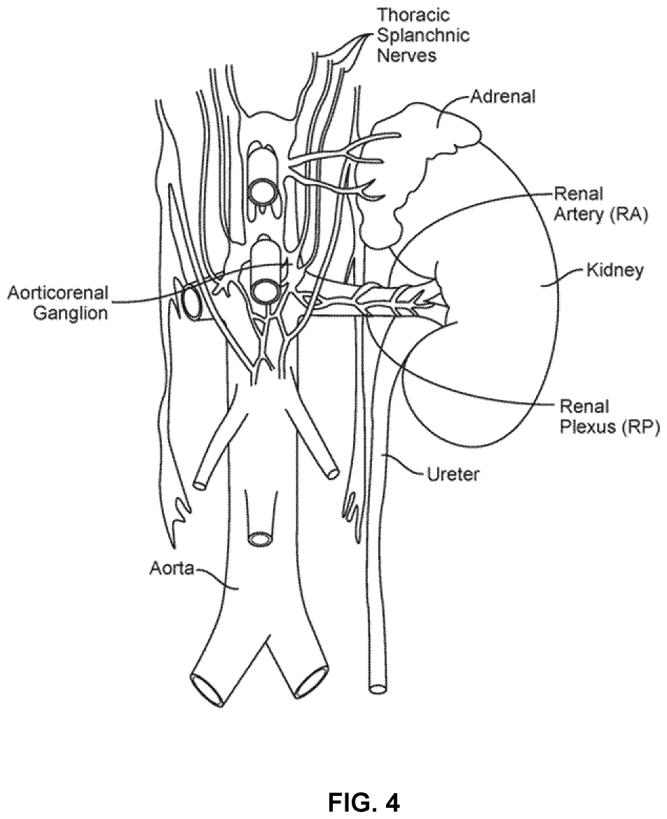

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease in some patients are characterized by heightened sympathetic nervous activation. In patients with end stage renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects and others.

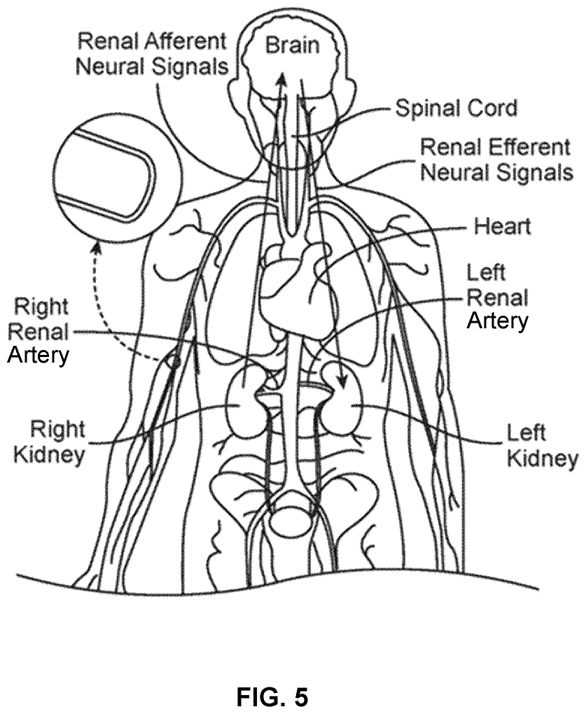

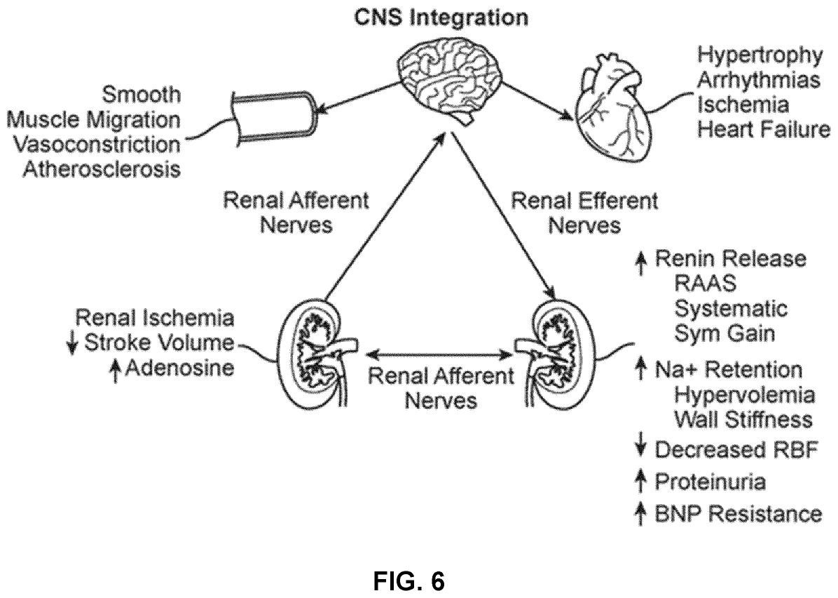

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

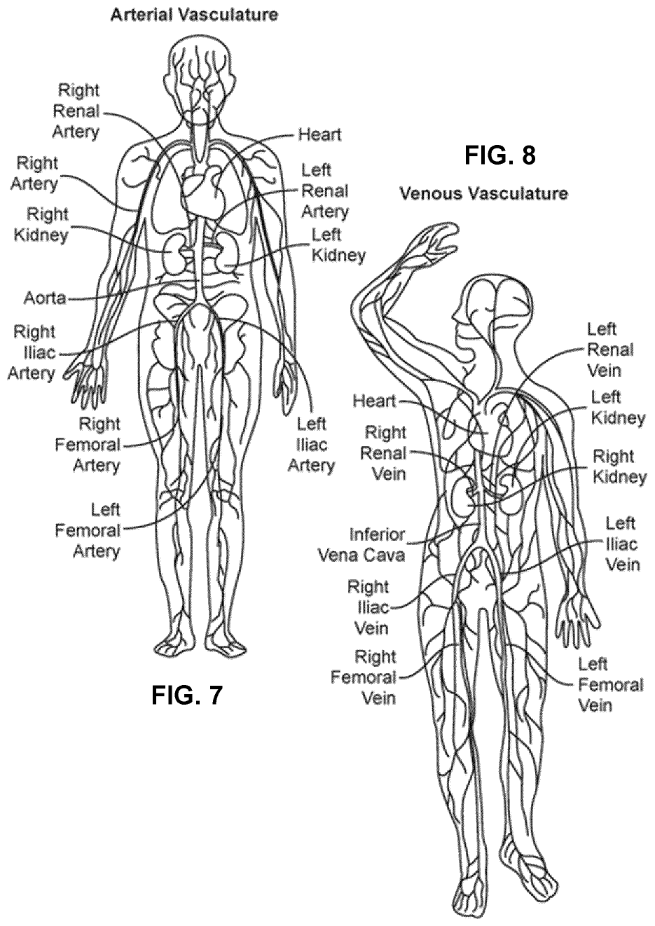

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. As discussed in greater detail below, the intima-media thickness separating the vessel lumen from its adventitia means that target renal nerves may be multiple millimeters distant from the luminal surface of the artery. The chemical agent should be delivered to the target renal nerves to modulate the target renal nerves without excessively adversely impacting the vessel wall to the extent that the vessel wall is potentially affected to an undesirable extent.

The neuromodulatory apparatus may also be configured to allow for adjustable positioning and repositioning of the therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 and/or expandable stabilizer 112 may intimately contact the vessel wall and/or extend at least partially through the vessel wall. Renal artery vessel diameter, D_{RA}, typically is in a range of about 2-10 millimeters (mm), with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the renal artery) to avoid non-target tissue and anatomical structures such as anatomical structures of the digestive system or psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between therapeutic elements 110 and/or expandable stabilizer 112 and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

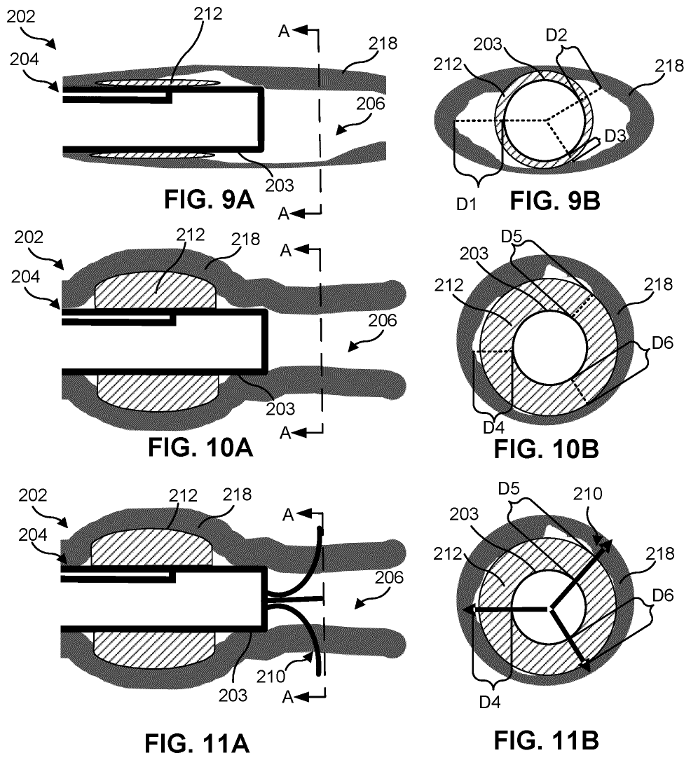

FIGS. 9A-11B are conceptual diagrams of an example neuromodulation catheter 202 that includes a distal portion 204 of an elongated shaft 203 of catheter 202 positioned within a renal artery 206. FIGS. 9A, 10A, and 11A are cross-sectional side views of catheter 202 illustrating a progression from a delivery configuration to a deployed configuration, and FIGS. 9B, 10B, and 11B are corresponding cross-sectional axial views of catheter 202 taken along line A-A (e.g., in a direction orthogonal to a longitudinal axis of a shaft of catheter 202). Neuromodulation catheter 202 is an example of neuromodulation catheter 102 of FIG. 1. In some instances, distal portion 204 may be positioned in a distal aspect of a main renal artery. Renal artery 206 includes vessel wall 218. Neuromodulation catheter 202 includes an expandable stabilizer 212 positioned circumferentially about distal portion 204. Elongated shaft 203 may define a lumen of neuromodulation catheter 202 at distal portion 204. In some examples, expandable stabilizer 212 may be a balloon, an expandable wire basket, or the like. In the example shown, expandable stabilizer 212 is an expandable balloon 212.

Neuromodulation catheter 202 is configured to deliver a chemical agent, such as a neurotoxic chemical, through the plurality of therapeutic elements 210 (shown in FIGS. 11A-11B). The chemical agent may be selected to neuromodulate (e.g., chemically ablate) nerve tissue of the renal plexus adjacent to renal artery 206. The chemical agent may include, for example, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof. In some examples, the chemical agent may be heated or cooled to additionally or alternatively thermally ablate nerve tissue of the renal plexus adjacent to renal artery.

In the example shown in FIGS. 11A-11B, three therapeutic elements 210 are shown. In other examples, neuromodulation catheter 202 includes only one or two therapeutic elements 210 or more than three therapeutic elements 210. Each therapeutic element 210 includes a corresponding needle and, in some, but not all, examples, a corresponding guide tube. Each guide tube is configured to, for example, define a pathway through which a corresponding needle may traverse to reach a target site (e.g., a location of the vessel wall) for delivery of a therapy. In the examples shown, the three therapeutic elements 210 are circumferentially equally spaced, e.g., configured to extend from positions separated by 120° about the circumference of elongated shaft 203 of distal portion 204.

As illustrated in FIGS. 9A-9B, therapeutic elements 210 and balloon 212 are configured to be carried by neuromodulation catheter 202 in a delivery configuration as distal portion 204 of neuromodulation catheter 202 is advanced through vasculature of the patient to renal artery 206. In the delivery configuration, the needles may be retracted and/or non-extended within their corresponding guide tubes, and the guide tubes may be retracted and/or non-extended within distal portion 204, e.g., within the lumen defined by elongated shaft 203 (and are not visible in FIGS. 9A-9B). Balloon 212 may be in an unexpanded and/or deflated configuration.

When distal portion 204 is positioned within renal artery 206 near the target treatment site, neuromodulation catheter 202 may be configured to transform to a deployed configuration. Near the target treatment site, and as illustrated in FIGS. 9A-9B, renal artery 206 may be a tortuous artery having bends and/or a non-uniform and/or asymmetric vessel wall 218 thickness that varies along its length (e.g., due to atherosclerotic plaque burden), and the lumen of renal artery 206 may be asymmetric. Generally, in transforming to a deployed configuration, the guide tubes may be radially extended to contact a luminal surface of renal artery 206 and stabilize distal portion 204. However, as shown in FIG. 9B, the radial distance for each of the three guide tubes to contact the luminal surface may be different, e.g., distances D1-D3 are different. The varying distances D1-D3 may increase the difficulty in accurately and precisely deploying the needles to the target treatment site because it may be difficult to radially extend each of the needles to the correct penetration depth (e.g., a uniform penetration depth with respect to vessel wall 218).

Balloon 212 may be configured to apply a force to vessel wall 218 to urge a portion of renal artery 206 contacted by balloon 212 to straighten relative to its un-contacted state. Balloon 212 alternatively and/or additionally may be configured to reduce a curvature of vessel wall 218 proximate the target treatment site via repositioning at least a portion of the length of the vessel wall proximate the target treatment site, as illustrated in FIGS. 10A-10B. Additionally, balloon 212 may be configured to substantially radially center distal portion 204 within renal artery 206 and to substantially maintain distal portion 204 substantially stationary relative to vessel wall 218, e.g., substantially stabilizing distal portion 204. Any or all of centering and stabilizing distal portion 204, straightening artery 206, and/or reducing the curvature of vessel wall 212 proximate the target treatment site may reduce the variation in distances D1-D3 the guide tubes and needles radially extend to penetrate vessel wall 218 and/or improve the accuracy and precision with which the needles may be positioned at the target treatment site to deliver the chemical agent.

FIGS. 10A-10B illustrate a first stage of neuromodulation catheter 202 transforming to a deployed configuration. In the example shown, balloon 212 is expanded and/or inflated to contact and apply a force to vessel wall 218. As illustrated in FIG. 10B, balloon 212 in the expanded/inflated configuration may reposition at least a portion of the length of vessel wall 218 proximate the target treatment site to straighten renal artery 206 and a reduce the longitudinal curvature of vessel wall 218, and to change the distances that the needle guides and needles radially extend to contact the luminal surface of vessel wall 218, e.g., from distances D1-D3 to distances D4-D6. In the example shown, the variation between distances D4-D6 is substantially less than D1-D3.

FIGS. 11A-11B illustrate a second stage of neuromodulation catheter 202 transforming to a deployed configuration. In the examples shown, therapeutic elements 210 (e.g., the guide tubes and needles) may be advanced out of neuromodulation catheter 202 to extend radially outwards relative to neuromodulation catheter 202. For example, guide tubes may be advanced out of neuromodulation catheter 202 to extend radially outwards relative to neuromodulation catheter 202 and contact vessel wall 218. After the guide tubes have been advanced out of neuromodulation catheter 202 to contact the wall 218, the needles may be advanced out of the guide tubes at least partially through wall 218 (e.g., only partially through wall 218 or completely through wall 218). Alternatively, the needles may be fixed relative to the guide tubes, such that the guide tubes and the needles are extended out of neuromodulation catheter 202 together and the exposed length of the needles out of the guide tubes is fixed. To transition back to the delivery configuration, therapeutic elements 210 (e.g., the guide tubes and needles) may be retracted within neuromodulation catheter 202, and balloon 212 may be unexpanded and/or deflated.

As another example, in the delivery configuration, the needles may be withdrawn into the guide tubes and the guide tubes may be urged against an outer surface of neuromodulation catheter 202 using a guide sheath (not shown). To transition to the deployed configuration, e.g., at the second stage the guide sheath may be withdrawn proximally relative to neuromodulation catheter 202, which allows the guide tubes to expand radially outward. As such, in some examples, the guide tubes may be formed of a resilient material, such as a shape memory material, which is self-expanding upon being released from the guide sheath. Once the guide tubes have expanded to contact vessel wall 218, the needles may be advanced out of the guide tubes at least partially through vessel wall 218.

In some examples, positioning of distal portion 204 and therapeutic elements 210 may be aided by medical imaging. To facilitate medical imaging of therapeutic elements 212, the guide tubes may include or be formed from a radiopaque material. For example, the guide tubes may be formed from a radiopaque metal, such as tantalum, gold, platinum, or the like. As another example, the guide tubes may be formed from a polymer and include a radiopaque marker element, such as a ring or band, formed from a radiopaque metal, such as tantalum, gold, platinum, barium, tungsten, or the like. As a further example, the guide tubes may be formed from a polymer filled with a radiopaque powder, such as tantalum, gold, platinum, barium, tungsten, or the like. In examples in which the guide tubes include a plastic, the plastic may include a polyamide, a polyurethane, or the like; or a multilayer construction that includes an inner layer of polyamide and an outer layer of polyurethane. Although a single balloon 212 is shown in FIGS. 9A-11B, in other examples, neuromodulation catheter 202 may include multiple balloons 212, e.g., one balloon 212 proximal of therapeutic elements 212 and one balloon 212 distal of therapeutic elements 212.

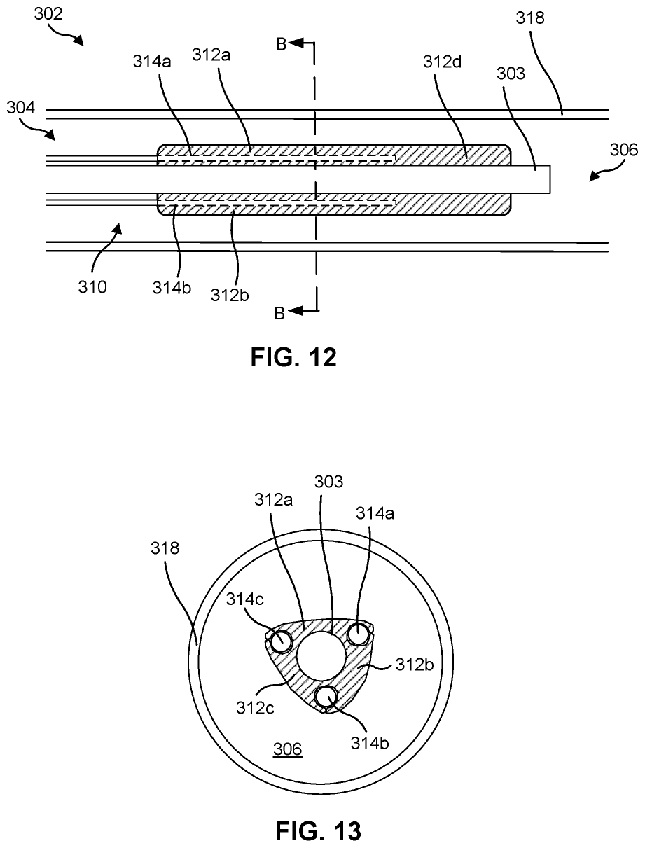

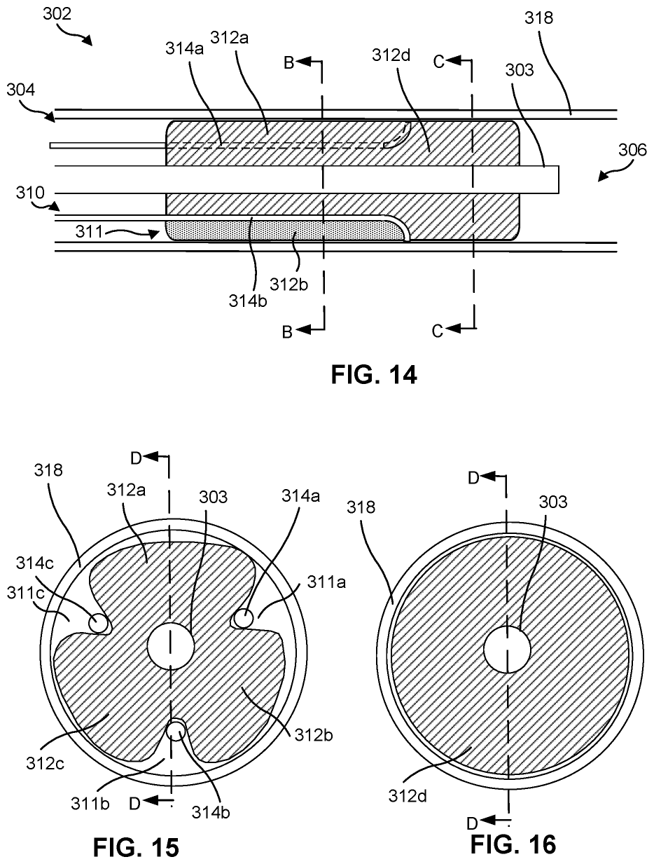

FIGS. 12-16 are conceptual diagrams of an example neuromodulation catheter 302 that includes a distal portion 304 of an elongated shaft of catheter 302 positioned within a renal artery 306. FIGS. 12 and 14 are cross-sectional side views of catheter 302 illustrating a delivery configuration (FIG. 12) and a partially deployed configuration (FIG. 14), e.g., taken along line D-D shown in FIGS. 15-16. FIG. 13 is a cross-sectional axial view taken along line B-B of catheter 302 in the delivery configuration corresponding to FIG. 12. FIG. 15 is a cross-sectional axial view taken along line B-B of catheter 302 in the partially deployed configuration of FIG. 14, and FIG. 16 is a cross-sectional axial view taken along line C-C of catheter 302, e.g., at a different longitudinal position of distal portion 304, in the partially deployed configuration of FIG. 14. Neuromodulation catheter 302 is an example of neuromodulation catheter 102 of FIG. 1. In some instances, distal portion 304 may be positioned in a distal aspect of a main renal artery, an accessory renal artery, a branch vessel of a main or accessory renal artery, or a renal vein. Renal artery 306 includes vessel wall 318.

Neuromodulation catheter 302 includes an expandable stabilizer 312 positioned circumferentially about distal portion 304 of elongated shaft 303. Elongated shaft 303 may define a lumen of neuromodulation catheter 302 at distal portion 302. Expandable stabilizer 312 includes a needle guide configured to position a distal end of the needles at a predetermined distance from a central axis of distal portion 304 when the expandable stabilizer 312 is in an expanded configuration and the needles are in the deployed configuration. Expandable stabilizer 312 may improve the accuracy and precision with which the needles may be positioned at the target treatment site to deliver the chemical agent. In some examples, expandable stabilizer 312 may be a balloon, an expandable wire basket, and the like.

As best seen in the example shown in FIG. 15, stabilizer 312 is a tri-lobed expandable balloon 312 including three lobed portions 312a, 312b, and 312c that extend for a portion of the longitudinal length of expanded balloon 312 and define three channels 311a, 311b, and 311c (collectively, channels 311) between respective lobes for the lobed portion of the longitudinal length of expanded balloon 312. The channels 311 may be needle guides, alternatively referred to as balloon guides, configured to guide the needles and/or guide tubes, e.g., to radially extend from distal portion 304. In other examples, expanded stabilizer 312 may include any number of needle guides and/or channels 311, e.g., expanded stabilizer 312 may include one channel 311, two channels 311, or four or more channels 311. In other examples, expanded stabilizer 312 may be a balloon including fewer or more than three lobes defining channels 311, e.g., two lobes or four or more lobes. In some examples, the lobes and channels 311 may extend the entire longitudinal length of expanded balloon 312. In some examples, expanded stabilizer 312 may be an expandable member other than a balloon including one or more needle guides which may or may not be channels, e.g., an expandable basket and the like.

Neuromodulation catheter 302 is configured to deliver a chemical agent, such as a neurotoxic chemical, through the plurality of therapeutic elements 310 similar to neuromodulation catheter 202 described above. As best seen in FIGS. 13 and 15, neuromodulation catheter 302 includes three therapeutic elements 310 (e.g., which include the three guide tubes 314a-314c which are visible in the examples shown). In other examples, neuromodulation catheter 302 includes only one or two therapeutic elements 310 or more than three therapeutic elements 310. Each therapeutic element 310 includes a corresponding needle and, in some, but not all, examples, a corresponding guide tube 314a, 314b, and 314c (collectively guide tubes 314). Each guide tube 314 is configured to, for example, define a pathway through which a corresponding needle may traverse to reach a target site for delivery of a therapy. In the examples shown, the three therapeutic elements 310 are circumferentially equally spaced, e.g., configured to extend from positions separated by 120° about the circumference of elongated shaft 303 of distal portion 304.

As illustrated in FIGS. 12-13, therapeutic elements 310 and expandable balloon 312 are configured to be carried by neuromodulation catheter 302 in a delivery configuration as distal portion 304 of neuromodulation catheter 302 is advanced through vasculature of the patient to renal artery 306. In the delivery configuration, the needles may be retracted and/or non-extended within their corresponding guide tubes 314. Expandable balloon 312 is in an unexpanded and/or deflated configuration. In contrast with neuromodulation catheter 202, guide tubes 314 may be external to a lumen of distal portion 304. In the example shown, guide tubes 314 are within channels 311 defined by expandable balloon 312 for a portion of the longitudinal length of expandable balloon 312, and are relatively parallel to a central axis of distal portion 304 in the delivery configuration. In the example shown, the lobes of expandable balloon 312 in the unexpanded/deflated delivery configuration circumferentially encompass guide tubes 314, and may function to protect guide tubes 314 during delivery and/or protect artery 306 and vessel wall 318 from damage due to contact with guide tubes 314.

Expandable balloon 312 may comprise a non-compliant and/or a semi-compliant material. For example, expandable balloon 312 may be configured with a durometer sufficient to also protect guide tubes 314 in the delivery configuration and to protect expandable balloon 312 in the delivery configuration, e.g., from damage by guide tubes 314. Expandable balloon 312 may be configured with a durometer sufficient to support and/or radially extend guide tubes 314 towards vessel wall 318, e.g., push guide tubes 314 out to angle guide tubes 314 away from the central axis of distal portion 304, during deployment.

For example, a portion of expandable balloon 312 that is not a lobe portion, e.g., balloon portion 312d as shown in FIGS. 14 and 16, may be configured to bend and/or angle guide tubes 314 away from the central axis of distal portion 304 to radially extend guide tubes 314, and to support guide tubes 314 while in the deployed configuration. Alternatively stated, balloon portion 312d may be considered a fourth balloon lobe 312d that is a single lobe about the circumference of distal portion 304 and, along with balloon lobes 312a, 312b, and 312d, define channels 311 that extend longitudinally and substantially parallel with the central axis of distal portion 304 for the longitudinal portion corresponding to lobes 312a-312c, and then bends and angles in the radial direction toward vessel wall 318 via balloon lobe 312d. For example, as shown in FIG. 14, a distal portion of guide tube 314c is bent and supported by balloon lobe 314d in the deployed configuration. A portion of an outer surface of balloon lobe 312b, defining a portion of the channel 311 in which guide tube 314b is positioned, is visible in the cross-sectional view of FIG. 14.

In some examples, guide tubes 314 may be loosely positioned within the channels/needle guides 311, e.g., not attached to balloon 314. In other examples, guide tubes 314 may be attached to balloon 314 within the channels 311, e.g., bonded, welded, glued, adhered with an adhesive, and the like.

Similar to neuromodulation catheter 202 described above, neuromodulation catheter 302 may be configured to apply a force to vessel wall 318 to urge a portion of renal artery 306 contacted by balloon 312 to straighten and to reduce a curvature of vessel wall 312 proximate the target treatment site via, as well as substantially radially center and stabilize distal portion 304 within renal artery 206. In the examples shown, neuromodulation catheter 302 may be further configured to circumferentially position and stabilize therapeutic elements 310, e.g., needle guides 314 and corresponding needles, relative to vessel wall 318. As illustrated in FIGS. 13 and 15, balloon 314 defines channels 311 configured to circumferentially position and hold needle guides 314 in the deployed configuration (FIG. 15) and, in some examples, the delivery configuration (FIG. 13). In some examples, channels 311 defined by balloon lobes 314a-314c may also radially position and hold needle guides 314 relative to vessel wall 318 and/or the central axis of distal portion 304, e.g., channels 311 may radially extend in the deployed configuration relative to the delivery configuration.

As described above, expandable balloon 312 is further configured to define one or more needle guides, e.g., channels 311, which are configured to position the distal ends of guide tubes 314 to contact vessel wall 318 or to be proximate to vessel wall 318, e.g., close to vessel wall 318 but not in contact with vessel wall 318. For example, guide tubes 314 may be configured to radially extend the same radial distance as the lobes of expandable balloon 312 are configured to extend, or less than the radial distance that the lobes of expandable balloon 312 are configured to extend. In other words, a radial extent of expandable balloon 312 may be greater than or equal to a radial extent of the distal end of guide tubes 314 when expandable balloon 312 is in the expanded configuration, and expandable balloon 312 may be configured to prevent and/or reduce puncturing of vessel wall 318 by guide tubes 314.

In some examples, expandable balloon 312 is configured to position guide tubes 314 to radially extend a predetermined distance, e.g., the same as or less than the radial extent of the lobes of expandable balloon 312 adjacent to the respective guide tube. In this way, expandable balloon 312 may be configured to position therapeutic elements 310, e.g., guide tubes 314 and their corresponding needles, such that the needles may extend a predetermined radial distance into vessel wall 318 and target tissue at the tarte treatment site. In some examples, the target tissue may be perivasculature adipose tissue proximate the vessel wall 318.

In some examples, neuromodulation catheter 302 may not include guide tubes 314, and may include the needles (e.g., guide tubes 314 may represent needles in the examples shown). Expandable balloon 312 may be configured to encompass and/or protect the needles in the delivery configuration as described above with reference to guide tubes 314, and may be configured to push and/or radially extend a distal end of the needles away from the central axis of distal portion 304 when expandable balloon 312 is in the expanded configuration.

Although the examples shown illustrate expandable balloon 312 as including multiple lobes for a portion of the longitudinal length of expandable balloon 312, in some examples balloon 314 may include multiple lobes for the entire longitudinal length of expandable balloon 312. For example, expandable balloon 312 may not include balloon lobe 312d, and channels 311 defined by balloon lobes 312a-312c may extend the entire longitudinal length of expandable balloon 312 and position and hold therapeutic elements 310, e.g., needle guides 314 and/or needles, circumferentially. The needles and/or needle guides 314 may then be configured to extend radially, e.g., when released by expandable balloon 312 during deployment. For example, the needles and/or needle guides 314 may be formed of a resilient material, such as a shape memory material, which is self-expanding upon being released from expandable balloon 312 (e.g., which may include overlapping balloon lobes 314a-314c in the delivery configured to hold the needles and/or needle guides 314) or some other delivery configuration maintaining element, such as a guide sheath.

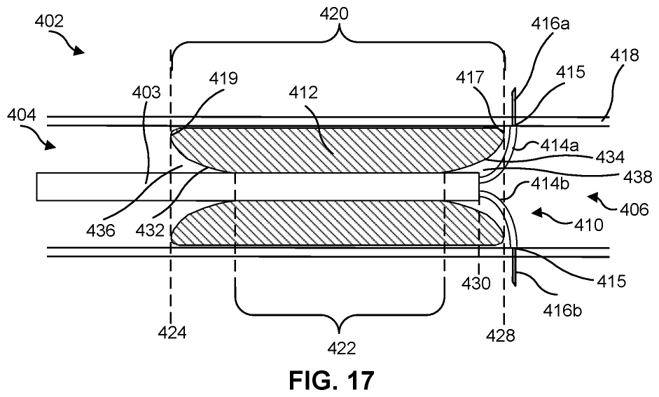

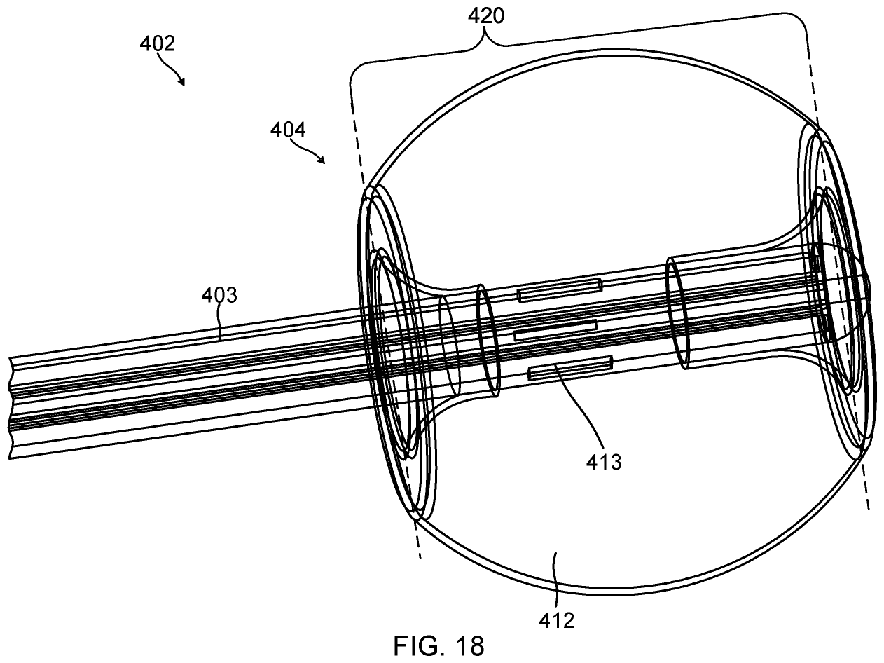

FIGS. 17 and 18 are conceptual diagrams of an example neuromodulation catheter 402 that includes a distal portion 404 of an elongated shaft 403 of catheter 402 positioned within a renal artery 406. FIG. 17 is a cross-sectional side view of catheter 402 illustrating a deployed configuration. FIG. 18 is a perspective view of catheter 402 with an expandable stabilizer 412 in an expanded configuration. Neuromodulation catheter 402 is an example of neuromodulation catheter 102 of FIG. 1. In some instances, distal portion 404 may be positioned in a distal aspect of a main renal artery. Renal artery 406 includes vessel wall 418.

Neuromodulation catheter 402 may be substantially similar to neuromodulation catheter 202, except that neuromodulation catheter 402 includes an expandable stabilizer 412 configured with a perimeter longitudinal (e.g., axial) length 420 at a radial perimeter of expandable stabilizer 412 that is greater than an inner longitudinal length 422 at a radially inwards position of the expandable stabilizer 412 proximate an outer surface of distal portion 402. Elongated shaft 403 may define a lumen of neuromodulation catheter 402 at distal portion 404.

Expandable stabilizer 412 may be configured to provide support and stabilization to vessel wall 418 substantially close to a puncture position 415 of needles 416. In some examples, puncture position 415 may correspond to a vessel wall contact, or near contact, position of guide tubes 414. For example, by comparison with expandable stabilizer 212 of FIGS. 9A-11B, stabilizer 414 may be configured to further improve reducing the variation in distances guide tubes 414 and/or needles 416 need to radially extend to contact/penetrate vessel wall 418 and improve the accuracy and precision with which needles 416 may be positioned at the target treatment site to deliver the chemical agent by reducing the longitudinal distance between puncture position 415 and the distal end 417 of expandable stabilizer 412, e.g., at distal plane 428 in the example shown. In other words, expandable stabilizer 412 is configured to provide radial stabilization of vessel wall 418 near to puncture position 415. For example, expandable stabilizer 412 is configured such that, when expanded and/or inflated, distal end 417 is less than or equal to 10 mm from puncture position 415, or distal end 417 is less than or equal to 1 mm from puncture position 415, or distal end 417 is substantially adjacent to puncture position 415. In some examples, expandable stabilizer 412 is configured to straighten artery 406, reduce the curvature of vessel wall 418, and/or stabilize therapeutic elements 410 substantially close and/or adjacent to the puncture/contact positions 415 of therapeutic elements 410. In the example shown, each of therapeutic elements 410 are configured to cause needles 416a and 416b to puncture vessel wall 418 at substantially the same longitudinal puncture position 415; however, in other examples, each of therapeutic elements 410 may be configured to cause needles 416a and 416b to puncture vessel wall 418 at different longitudinal puncture positions. For example, expandable stabilizer 412 may be configured to have a distal end 417 that varies in longitudinal position around the circumference of the inner surface of vessel wall 418, and configured to provide radial stabilization at or near, e.g., adjacent to or less than or equal to 10 mm or 1 mm, from the puncture positions of each of the therapeutic elements 410.

In the example shown, expandable stabilizer 412 is an expandable inverted neck balloon 412. Inverted neck balloon 412 may be attached to elongated shaft 403, e.g., along an outer surface of elongated shaft 403. In the example, shown, expandable stabilizer 412 is attached to an outer surface of elongated shaft 403 at least along a portion of an inner radial length 422 of expandable stabilizer 412. The inner radial length 422 of expandable stabilizer 412, when expanded/inflated, is less than the radial perimeter length 420 of expandable stabilizer 412. In the example shown, an outer surface 432 of expandable stabilizer 412, when expandable stabilizer 412 is expanded/inflated, extends radially outwards from the outer surface of elongated shaft 403 as well as in the proximal direction, and defines a proximal volume 436 within renal artery 406 between outer surface 432, the outer surface of elongated shaft 403, and distal to the proximal end 419 of expandable stabilizer 412, e.g., distal to proximal plane 424 in the example shown. In the example shown, an outer surface 434 of expandable stabilizer 412, when expandable stabilizer 412 is expanded/inflated, extends radially outwards from the outer surface of elongated shaft 403 as well as in the distal direction, and defines a distal volume 438 within renal artery 406 between outer surface 434, the outer surface of elongated shaft 403, and proximal to the distal end 417 of expandable stabilizer 412, e.g., proximal to distal plane 428 in the example shown.

In the example shown, distal end 430 of elongated shaft 403 is proximal to the distal end 417 of expandable stabilizer 412 and distal plane 428. Therapeutic elements 410 (which may comprise just needles 416, just needle guides 414 configured to house and guide needles 416, and/or needles 416 housed within needle guides 414) extend from distal end 430 of elongated shaft 403, e.g., from within distal volume 438. In other examples, distal end 430 may be distal to distal end 417 of expandable stabilizer 412 and/or distal plane 428. In such examples, therapeutic elements 410 may still extend from elongated shaft 403 from within distal volume 438, e.g., out of a sidewall and/or outer surface of elongated shaft 403. For example, whether distal end 430 of elongated shaft 403 is within distal volume 438 or distal to distal volume 438, expandable stabilizer 412, when expanded/inflated, and therapeutic elements 410 are be configured such that therapeutic elements 410 extend away from the central axis of the vessel (e.g., to extend in a radial direction away from a central axis of renal artery 406 and/or elongated shaft 403) within distal volume 438 and to puncture vessel wall 418 distal to distal end 417 of expandable stabilizer 412, e.g., at puncture position 415. In other words, expandable stabilizer 412 and therapeutic elements 410 may be configured such that therapeutic elements 410 begin to extend away from elongated shaft 403 proximal to the distal end of expandable stabilizer 412 at its radial perimeter so as to position needles 416 to puncture vessel wall 418 at or near distal end 417 of expandable stabilizer 412.n the example shown, needles 416 are configured to puncture vessel wall 418 proximate the radial perimeter of expandable stabilizer 412, e.g., at puncture position 415. In the example shown, therapeutic elements 410, e.g., guide tubes 414 and/or needles 416, are configured to extend away from the central axis of distal portion 404 within at least a portion of the perimeter longitudinal length 420 of the expandable stabilizer 412. For example, expandable stabilizer 412 is configured to allow therapeutic elements 410 to extend away from the central axis of distal portion 404, and towards vessel wall 418, while still proximal to distal end 417 of expandable stabilizer 412, e.g., within the perimeter longitudinal length 420 of expandable stabilizer 412 so as to contact and/or puncture vessel wall 418 at puncture position 415 just distal to distal end 417 of expandable stabilizer 412, e.g., just distal to the longitudinal length of expandable stabilizer 412 at its radial perimeter. In some examples, expandable stabilizer 412 may be a balloon, an expandable wire basket, or the like.

Neuromodulation catheter 402 is configured to deliver a chemical agent, such as a neurotoxic chemical, through the plurality of therapeutic elements 410 similar to neuromodulation catheters 202 and 302 described above. In the example shown, neuromodulation catheter 402 includes two therapeutic elements 410. In other examples, neuromodulation catheter 402 includes one therapeutic element 410 or more than two therapeutic elements 410. Each therapeutic element 410 includes a corresponding needle 416 and, in some, but not all, examples, a corresponding guide tube 414. Each guide tube 414 is configured to, for example, define a pathway through which a corresponding needle 416 may traverse to reach a target site for delivery of a therapy. In some examples, three therapeutic elements 410 may be circumferentially equally spaced, e.g., configured to extend from positions separated by 1200 about the circumference of elongated shaft 403 of distal portion 404.

As with neuromodulation catheters 202 and 302, therapeutic elements 410 and inverted neck balloon 412 are configured to be carried by neuromodulation catheter 402 in a delivery configuration (not shown) as distal portion 404 of neuromodulation catheter 402 is advanced through vasculature of the patient to renal artery 406. In the delivery configuration, the needles 416 may be retracted and/or non-extended within their corresponding guide tubes 414 and guide tubes 414 may be retracted and/or non-extended within distal portion 204, e.g., within the lumen defined by elongated shaft 403. When distal portion 404 is positioned within renal artery 406 near the target treatment site, neuromodulation catheter 402 may be configured to transform to a deployed configuration as illustrated in FIG. 17.

In some examples, elongated shaft 403 and distal portion 404 may include one or more inflation lumens (not shown) configured to deliver a fluid to and from inverted neck balloon 412, e.g., to inflate and/or deflated inverted neck balloon 412. The one or more inflation lumens may be fluidically coupled to a fluid source and/or sink (e.g., an air or saline tank) and/or a fluid motive source (e.g., an air or saline pump), e.g., at a proximal end of elongated shaft 403 and/or a proximal end of neuromodulation catheter 102, and/or handle 106, each of which may be connected to elongated shaft 403. In the example shown, distal portion 404 includes one or more fluid ports 413 (shown in FIG. 18). Fluid ports 413 are configured to be fluidically coupled to the one or more inflation lumens and configured to deliver, introduce, and/or remove a fluid between an interior of inverted neck balloon 412 and the one or more inflation lumens.

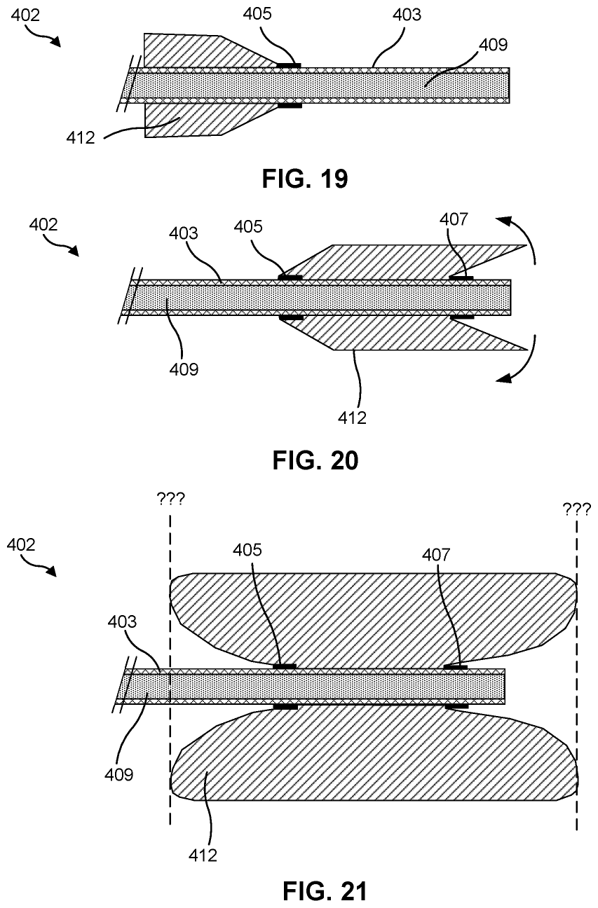

FIGS. 19-21 are conceptual cross-sectional side views of catheter 402 illustrating a progression of manufacturing and/or attaching inverted neck balloon 412 to elongated shaft 403. In the progression shown, a first (e.g., proximal) end of balloon 412 may be bonded, welded, adhered, and the like, to an outer surface of elongated shaft 403 at a first (e.g., more proximal) longitudinal position 405. Balloon 412 may then be folded over or inverted in the longitudinal direction as shown in FIG. 20. The second (e.g., distal) end of the inverted balloon 412 may then be bonded, welded, adhered, and the like, to an outer surface of elongated shaft 403 at a second (e.g., more distal) longitudinal position 407.

In some examples, any of expandable stabilizers and/or expandable balloons 112, 212, 312, and 412 described herein may comprise a low durometer polyurethane or Pebax®. For example, expandable stabilizers 112-412 may be configured to be formed with a diameter substantially similar to the outer diameter of respective elongated shafts 203-403 in the delivery configuration, e.g., to maintain a compact or low profile for delivery configuration, and to sufficiently expand to contact and/or exert a radial force on a vessel wall in the deployed and/or inflated configuration.

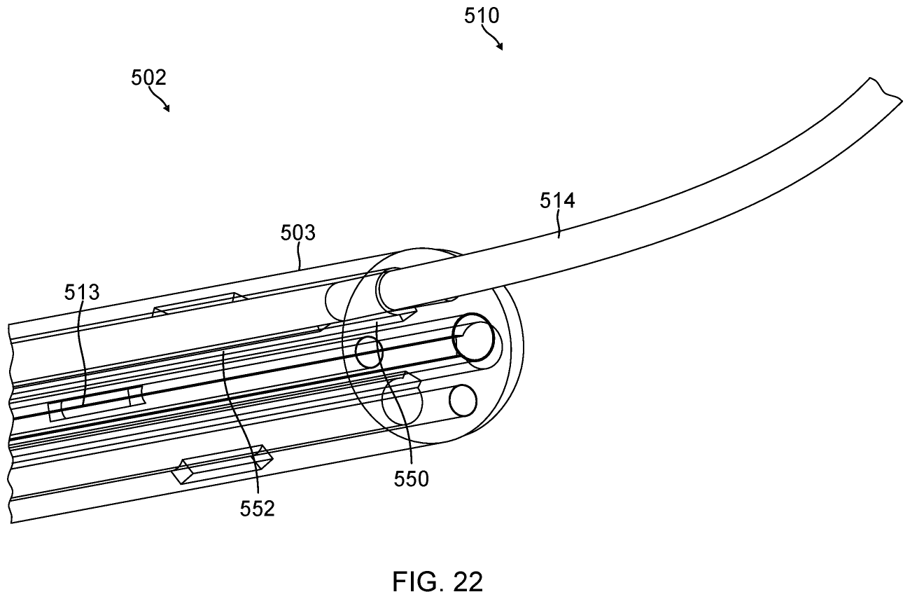

FIG. 22 is a conceptual perspective view of a neuromodulation catheter 502 illustrating a notch-and-groove mechanical guidance feature of distal portion 504. Neuromodulation catheter 502 is an example of neuromodulation catheter 102 of FIG. 1. In some instances, distal portion 504 may be positioned in a distal aspect of a main renal artery, and may be substantially similar to any of distal portions 204-404 described above.

In the example shown, elongated shaft 503 includes a plurality of lumens each configured to house a guide tube 514 or, in some examples, a needle. One or more of the lumens includes a recess 550, e.g., a groove, channel, slot, and the like, for at least a portion of its longitudinal length. The corresponding guide tube 514 (or needle) within the lumen includes a protrusion 552, e.g., notch, bump, and the like, configured to mate with the recess 550 and configured to reduce or substantially prevent the guide tube 514 from rotating about a longitudinal axis defined by the lumen. Protrusion 552 may be configured to move longitudinally within recess 550, e.g., to slide within the lumen. Recess 550 and protrusion 552 may be configured to reduce or substantially prevent therapeutic elements 510 from rotating and/or twisting when radially extended to contact and/or puncture a vessel wall. In some examples, distal portion 504 includes one or more fluid ports 513 configured to be fluidically coupled to the one or more inflation lumens (not shown) and configured to deliver, introduce, and/or remove a fluid between an interior of an expandable stabilizer and the one or more inflation lumens. Fluid ports 513 may be substantially similar to fluid ports 413 of FIG. 18 described above.

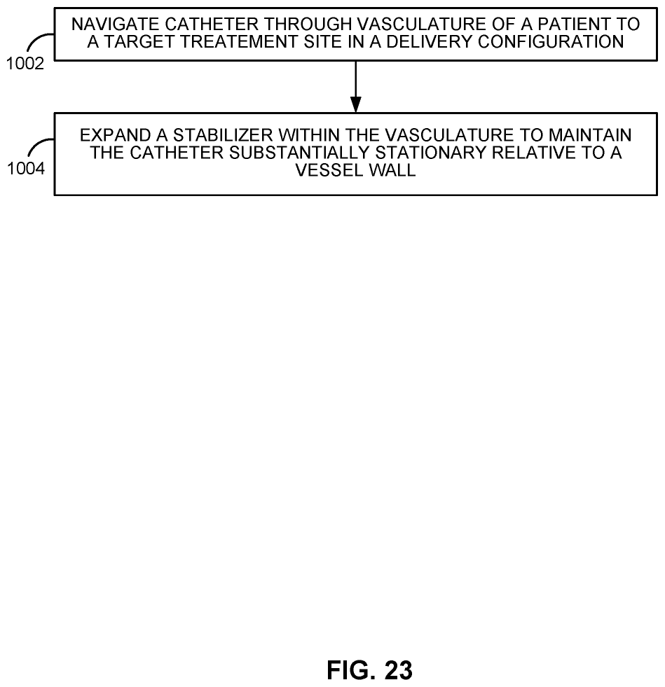

FIG. 23 is a flow diagram illustrating an example technique of performing neuromodulation of renal nerves in accordance with some examples of this disclosure. The technique of FIG. 23 will be described with concurrent reference to any or all of neuromodulation catheters 202-502 described above, although it will be appreciated that the technique of FIG. 23 may be performed with other neuromodulation catheters. Conversely, it will also be appreciated that neuromodulation catheters 202-502 may be used in other techniques.

The technique of FIG. 23 includes navigating a catheter through vasculature of a patient to a target treatment site in a vessel of the patient (1002). A clinician may access the renal vessel of the patient through an intravascular path, such as a percutaneous access site in the femoral, brachial, radial, or axillary artery to a targeted treatment site within the renal vessel. In some examples, the renal vessel may be a main renal artery, and the main renal artery may be adjacent a second renal vessel, such as an accessory renal artery. By manipulating a proximal portion of neuromodulation catheter 302 from outside the intravascular path, a clinician may advance at least distal portion 304 through the sometimes tortuous intravascular path and remotely manipulate distal portion 304.

The catheter may be any catheter described herein, for example, any of neuromodulation catheters 202-502. For example, the catheter may be catheter 302 and may comprise an elongated member of catheter 302, configured to be navigated through vasculature of a patient to the target treatment site, a needle positioned at a distal portion 304 of the elongated member, the needle configured to be within distal portion 304 when in a delivery configuration and radially extended to puncture a vessel wall of the vessel at the target treatment site when in a deployed configuration, and an expandable balloon 312 configured to guide the needle in the deployed configuration, balloon 312 including a needle guide and/or channel 311 configured to position a distal end of the needle at a predetermined distance from a central axis of distal portion 304 when balloon 312 is in the expanded configuration and the needle is in the deployed configuration.

The balloon may be expanded within the vasculature to maintain the elongated shaft 303, e.g., distal portion 304, substantially stationary relative to the vessel wall (1004). For example, a clinician may activate and/or manipulate catheter 302 and/or a component of system 100 to inflate expandable balloon 312, e.g., by introducing a fluid into an interior of expandable balloon 312. In some examples, expanding expandable balloon 312 may angle the distal end of the needle away from the central axis of distal portion 304 when expandable balloon 312 is in the expanded configuration. In some examples, expandable balloon 312 may include a plurality of lobes 312a-312d in the expanded configuration and a needle guide of neuromodulation catheter 302 may comprises a channel 311 between a first lobe of the plurality of lobes and a second lobe of the plurality of lobes, e.g., upon expanding the balloon to form the channel 311.

In some examples, expanding balloon 312 may substantially radially center the distal portion 304 within the vessel. In some examples, balloon 312 is configured to prevent a guide tube from puncturing the vessel wall when balloon 312 is in the expanded configuration. For example, the radial extent of balloon 312 in the expanded configuration may be greater than or equal to a radial extent of the distal end of the guide tube.

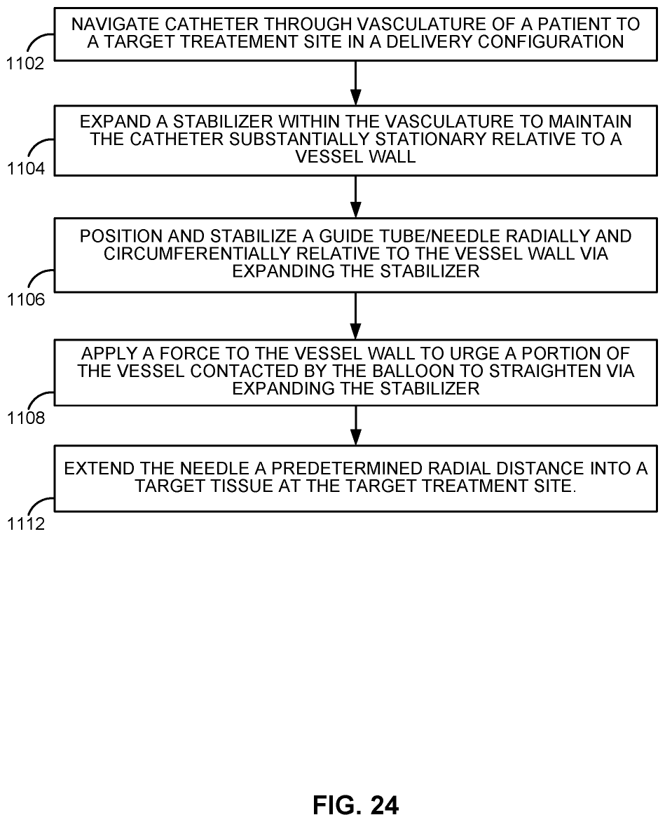

FIG. 24 is a flow diagram illustrating an example technique of performing neuromodulation of renal nerves in accordance with some examples of this disclosure. The technique of FIG. 24 will be described with concurrent reference to any or all of neuromodulation catheters 202-502 described above, although it will be appreciated that the technique of FIG. 24 may be performed with other neuromodulation catheters. Conversely, it will also be appreciated that neuromodulation catheters 202-502 may be used in other techniques.

The technique of FIG. 24 includes navigating a catheter through vasculature of a patient to a target treatment site in a vessel of the patient (1102), and expanding the balloon within the vasculature to maintain the elongated member, e.g., distal portion 304, substantially stationary relative to the vessel wall (1104), similar to that described above at (1002) and (1004), respectively.

A clinician may cause the balloon to expand, thereby causing the balloon to position and stabilize a guide tube and/or needle radially and circumferentially relative to the vessel wall within the vessel (1106). For example, a needle guide and/or needle may be radially and circumferentially positioned, supported, and stabilized in a channel 311 (e.g., needle guide) formed by one or more lobes of balloon 314.

A clinician may cause the balloon to expand, thereby causing the balloon to apply a force to the vessel wall to urge a portion of the vessel contacted by the balloon to straighten (1108). For example, balloon 212 may be expanded to straighten and reduce a curvature of vessel wall 212 proximate the target treatment site via repositioning at least a portion of the length of the vessel wall proximate the target treatment site, as illustrated in FIGS. 10A-10B. Balloon 212 may further, via expanding, center and stabilize distal portion 204 relative to vessel wall 218 and/or a central axis of distal portion 204, and reduce the variation in distances that guide tubes and/or needles need to radially extend to penetrate vessel wall 218 and thereby improve the accuracy and precision with which the needles may be positioned at the target treatment site to deliver a chemical agent.

A clinician may extend the needle a predetermined radial distance into a target tissue at the target treatment site (1110). For example, guide tubes 314 may be positioned and stabilized by balloon 312, and the clinician may advance needles through guide tubes 314 by a predetermine distance which may directly correspond to the distance that the needles penetrate into vessel wall 318, e.g., by virtue of more accurately positioning guide tubes 314 both circumferentially and radially via needle guides of balloon 312.

In some examples, the balloon may be an inverted neck balloon, e.g., inverted neck balloon 412. The inverted neck balloon 412 may have a perimeter longitudinal length at a radial perimeter of the inverted neck balloon 412 that is greater than an inner longitudinal length at a radially inwards position of the inverted neck balloon 412 proximate the distal portion 404 and/or housing 403. In some examples, the needle (e.g., needle 416) may be extended at (1110) away from the central axis of the vessel 406 within at least a portion of the perimeter longitudinal length of the inverted neck balloon 412. In some examples, the needle (e.g., needle 416) may be extended at (1110) to puncture the vessel wall 418 proximate the radial perimeter of the inverted neck balloon 412.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on delivering chemical agents, the present technology can equally be applied to other methods of neuromodulation therapy, including cooling, heating, electrical stimulation (using needle electrodes), RF energy delivery (using needle electrodes), or the like.

Certain aspects of the present disclosure described in the context of particular examples may be combined or eliminated in other embodiments. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Further disclosed herein is the subject-matter of the following clauses:

1. A catheter comprising:
an elongated member configured to be navigated through vasculature of a patient to a target treatment site in a vessel of the patient;
a needle positioned at a distal portion of the elongated member, the needle configured to be within the elongated member when in a delivery configuration and radially extended to puncture a vessel wall of the vessel at the target treatment site when in a deployed configuration; and
an expandable balloon including a needle guide configured to position a distal end of the needle at a predetermined distance from a central axis of the elongated member when the balloon is in an expanded configuration and the needle is in the deployed configuration.

2. The catheter of clause 1, wherein the balloon is configured to angle the distal end of the needle away from the central axis of the elongated member when the balloon is in the expanded configuration.

3. The catheter of clause 1 or clause 2, wherein the balloon comprises a plurality of lobes in the expanded configuration, and wherein the needle guide comprises a channel between a first lobe of the plurality of lobes and a second lobe of the plurality of lobes.

4. The catheter of any one of clauses 1 through 3, wherein, when the balloon is in the expanded configuration, the balloon is configured maintain the elongated member substantially stationary relative to the vessel wall.

5. The catheter of any one of clauses 1 through 4, wherein the balloon is configured to substantially radially center the elongated member within the vessel.

6. The catheter of any one of clauses 1 through 5, wherein the needle is configured to extend a predetermined radial distance into a target tissue at the target treatment site.

7. The catheter of clause 6, wherein the target tissue is a perivasculature adipose tissue proximate the vessel wall.

8. The catheter of any one of clauses 1 through 7, wherein the catheter further comprises a guide tube, wherein, when the balloon is in the expanded configuration, the needle guide is configured to radially and circumferentially position the guide tube relative to the vessel wall, wherein, when the balloon is in the expanded configuration, the needle guide is configured to stabilize the guide tube from moving circumferentially or radially relative to the vessel wall, and wherein the needle is configured to extend from the guide tube.

9. The catheter of clause 8, wherein balloon is configured to prevent the guide tube from puncturing the vessel wall when the balloon is in the expanded configuration.

10. The catheter of clause 9, wherein a radial extent of the balloon is greater than or equal to a radial extent of the distal end of the guide tube when the balloon is in the expanded configuration.

11. The catheter of any one of clauses 1 through 10, wherein, when the balloon is in the expanded configuration, the balloon is configured to apply a force to the vessel wall to urge a portion of the vessel contacted by the balloon to straighten.

12. The catheter of clause 11, wherein, when the balloon is in the expanded configuration, the balloon is configured to reduce a curvature of the vessel wall proximate the target treatment site via repositioning at least a portion of the length of the vessel wall proximate the target treatment site.

13. The catheter of any one of clauses 1 through 12, where in the balloon comprises an inverted neck balloon.

14. The catheter of clause 13, wherein the inverted neck balloon comprises a perimeter longitudinal length at a radial perimeter of the inverted neck balloon that is greater than an inner longitudinal length at a radially inwards position of the inverted neck balloon proximate the elongated member.

15. The catheter of clause 14, wherein the needle is configured to extend away from the central axis of the vessel within at least a portion of the perimeter longitudinal length of the inverted neck balloon.

16. The catheter of clause 15, wherein the needle is configured to puncture the vessel wall proximate the radial perimeter of the inverted neck balloon.

17. A method comprising:
navigating a catheter through vasculature of a patient to a target treatment site in a vessel of the patient, wherein the catheter comprises:
   an elongated member configured to be navigated through vasculature of a patient to the target treatment site;
   a needle positioned at a distal portion of the elongated member, the needle configured to be within the elongated member when in a delivery configuration and radially extended to puncture a vessel wall of the vessel at the target treatment site when in a deployed configuration; and
   an expandable balloon including a needle guide configured to position a distal end of the needle at a predetermined distance from a central axis of the elongated member when the balloon is in an expanded configuration and the needle is in the deployed configuration; and
expanding the balloon within the vasculature to maintain the elongated member substantially stationary relative to the vessel wall.

18. The method of clause 17, wherein expanding the balloon angles the distal end of the needle away from the central axis of the elongated member when the balloon is in the expanded configuration.

19. The method of clause 17 or clause 18, wherein the balloon comprises a plurality of lobes in the expanded configuration, and wherein the needle guide comprises a channel between a first lobe of the plurality of lobes and a second lobe of the plurality of lobes, wherein expanding the balloon forms the channel.

20. The method of any one of clauses 17 through 19, wherein expanding the balloon substantially radially centers the elongated member within the vessel.

21. The method of any one of clauses 17 through 20, further comprising extending the needle a predetermined radial distance into a target tissue at the target treatment site.

22. The method of clause 21, wherein the target tissue is a perivasculature adipose tissue proximate the vessel wall.

23. The method of any one of clauses 17 through 21, further comprising:
positioning, via expanding the balloon, a guide tube radially and circumferentially relative to the vessel wall within the vessel; and
stabilizing, via expanding the balloon, the guide tube from moving radially and circumferentially relative to the vessel wall, and wherein the needle is configured to extend from the guide tube.

24. The method of clause 23, wherein balloon is configured to prevent the guide tube from puncturing the vessel wall when the balloon is in the expanded configuration.

25. The method of clause 24, wherein a radial extent of the balloon is greater than or equal to a radial extent of the distal end of the guide tube when the balloon is in the expanded configuration.

26. The method of any one of clauses 17 through 25, further comprising applying a force to the vessel wall, via expanding the balloon, to urge a portion of the vessel contacted by the balloon to straighten.

27. The method of clause 26, when the balloon is in the expanded configuration, the balloon is configured to reduce a curvature of the vessel wall proximate the target treatment site via repositioning at least a portion of the length of the vessel wall proximate the target treatment site.

28. The method of any one of clauses 17 through 27, where in the balloon comprises an inverted neck balloon.

29. The method of clause 28, wherein the inverted neck balloon comprises a perimeter longitudinal length at a radial perimeter of the inverted neck balloon that is greater than an inner longitudinal length at a radially inwards position of the inverted neck balloon proximate the elongated member.

30. The method of clause 29, further comprising extending the needle away from the central axis of the vessel within at least a portion of the perimeter longitudinal length of the inverted neck balloon.

31. The method of clause 30, further comprising puncturing the vessel wall proximate the radial perimeter of the inverted neck balloon.

32. The method of any one of clauses 17 through 31, further comprising:
extending the needle radially relative to the central axis of the elongated member; and
puncturing the vessel wall at the target treatment site.

33. A chemical ablation system comprising:
an ablation catheter comprising:
   an elongated member configured to be navigated through vasculature of a patient to a target treatment site in a vessel of the patient;
   a needle positioned at a distal portion of the elongated member, the needle configured to be within the elongated member when in a delivery configuration and radially extended to puncture a vessel wall of the vessel at the target treatment site when in a deployed configuration; and
   an expandable balloon including a needle guide configured to position a distal end of the needle at a predetermined distance from a central axis of the elongated member when the balloon is in an expanded configuration and the needle is in the deployed configuration; and
a medical device communicatively coupled to a lumen of the needle and configured to control delivery of a fluid via the lumen to the target treatment site.

34. A catheter comprising:
an elongated member configured to be navigated through vasculature of a patient to a target treatment site in a vessel of the patient;
a needle positioned at a distal portion of the elongated member, the needle configured to be within the elongated member when in a delivery configuration and radially extended to puncture a vessel wall of the vessel at the target treatment site when in a deployed configuration; and
an expandable inverted neck balloon comprising:
   when in an expanded configuration, a perimeter longitudinal length at a radial perimeter of the inverted neck balloon that is greater than an inner longitudinal length at a radially inwards position of the inverted neck balloon proximate the elongated member,
wherein the expandable inverted neck balloon is configured to position a distal end of the needle at a predetermined distance from a central axis of the elongated member when the balloon is in the expanded configuration and the needle is in the deployed configuration.

35. The catheter of clause 34, wherein the expandable inverted neck balloon is attached to the elongated member at a first distal position proximal the distal end of the expandable inverted neck balloon, wherein the distal end of the expandable inverted neck balloon, when inflated, is distal to the distal end of the elongated member and radially outwards from the first distal position.

36. The catheter of clause 34 or clause 35, wherein the inverted neck balloon is configured to provide radial stabilization to a vessel wall proximate to target the treatment site and at the distal end of the expandable inverted neck balloon, wherein the needle is configured to puncture the vessel wall proximate the distal end of the expandable inverted neck balloon.

37. The catheter of any one of clauses 34 through 36, wherein the needle is configured to extend away from the central axis of the vessel proximal to the distal end of the expandable inverted neck balloon and puncture the vessel wall distal to the distal end of the expandable inverted neck balloon.

38. The catheter of any one of clauses 34 through 37, wherein, when the balloon is in the expanded configuration, the balloon is configured maintain the elongated member substantially stationary relative to the vessel wall.

39. The catheter of any one of clauses 34 through 38, wherein the balloon is configured to substantially radially center the elongated member within the vessel.

40. The catheter of any one of clauses 34 through 39, wherein the needle is configured to extend a predetermined radial distance into a target tissue at the target treatment site.

41. The catheter of clause 40, wherein the target tissue is a perivasculature adipose tissue proximate the vessel wall.

42. The catheter of any one of clauses 34 through 41, wherein, when the balloon is in the expanded configuration, the balloon is configured to apply a force to the vessel wall to urge a portion of the vessel contacted by the balloon to straighten.

43. The catheter of clause 42, wherein, when the balloon is in the expanded configuration, the balloon is configured to reduce a curvature of the vessel wall proximate the target treatment site via repositioning at least a portion of the length of the vessel wall proximate the target treatment site.

44. A catheter comprising:
an elongated member configured to be navigated through vasculature of a patient to a target treatment site in a vessel of the patient;
a needle positioned at a distal portion of the elongated member, the needle configured to be within the elongated member when in a delivery configuration and radially extended to puncture a vessel wall of the vessel at the target treatment site when in a deployed configuration; and
an expandable balloon configured to position a distal end of the needle at a predetermined distance from a central axis of the elongated member when the balloon is in an expanded configuration and the needle is in the deployed configuration,
wherein, when the balloon is in the expanded configuration, the balloon is configured to apply a force to the vessel wall to urge a portion of the vessel contacted by the balloon to straighten.

45. The catheter of clause 44, wherein, when the balloon is in the expanded configuration, the balloon is configured to reduce a curvature of the vessel wall proximate the target treatment site via repositioning at least a portion of the length of the vessel wall proximate the target treatment site.

46. The catheter of clause 44 or clause 45, wherein, when the balloon is in the expanded configuration, the balloon is configured maintain the elongated member substantially stationary relative to the vessel wall.

47. The catheter of any one of clauses 44 through 46, wherein, when the balloon is in the expanded configuration, the balloon is configured to substantially radially center the elongated member within the vessel.

48. The catheter of any one of clauses 44 through 47, wherein the needle is configured to extend a predetermined radial distance into a target tissue at the target treatment site.

49. The catheter of clause 48, wherein the target tissue is a perivasculature adipose tissue proximate the vessel wall.

50. The catheter of any one of clauses 44 through 49, where in the balloon comprises an inverted neck balloon.

51. The catheter of clause 50, wherein the inverted neck balloon comprises a perimeter longitudinal length at a radial perimeter of the inverted neck balloon that is greater than an inner longitudinal length at a radially inwards position of the inverted neck balloon proximate the elongated member.

52. The catheter of clause 51, wherein the needle is configured to extend away from the central axis of the vessel within at least a portion of the perimeter longitudinal length of the inverted neck balloon.

53. The catheter of clause 52, wherein the needle is configured to puncture the vessel wall proximate the radial perimeter of the inverted neck balloon.

1. A catheter comprising:
an elongated member configured to be navigated through vasculature of a patient to a target treatment site in a vessel of the patient;
a needle positioned at a distal portion of the elongated member, the needle configured to be within the elongated member when in a delivery configuration and radially extended to puncture a vessel wall of the vessel at the target treatment site when in a deployed configuration; and
an expandable balloon including a needle guide configured to position a distal end of the needle at a predetermined distance from a central axis of the elongated member when the balloon is in an expanded configuration and the needle is in the deployed configuration.

2. The catheter of claim 1, wherein the balloon is configured to angle the distal end of the needle away from the central axis of the elongated member when the balloon is in the expanded configuration.

3. The catheter of claim 1, wherein the balloon comprises a plurality of lobes in the expanded configuration, and wherein the needle guide comprises a channel between a first lobe of the plurality of lobes and a second lobe of the plurality of lobes.

4. The catheter of claim 1, wherein the needle is configured to extend a predetermined radial distance into a target tissue at the target treatment site.

5. The catheter of claim 1, wherein the catheter further comprises a guide tube, wherein, when the balloon is in the expanded configuration, the needle guide is configured to radially and circumferentially position the guide tube relative to the vessel wall, wherein, when the balloon is in the expanded configuration, the needle guide is configured to stabilize the guide tube from moving circumferentially or radially relative to the vessel wall, and wherein the needle is configured to extend from the guide tube.

6. The catheter of claim 5, wherein balloon is configured to prevent the guide tube from puncturing the vessel wall when the balloon is in the expanded configuration.

7. The catheter of claim 1, wherein, when the balloon is in the expanded configuration, the balloon is configured to apply a force to the vessel wall to urge a portion of the vessel contacted by the balloon to straighten.

8. The catheter of claim 1, where in the balloon comprises an inverted neck balloon.

9. The catheter of claim 8, wherein the inverted neck balloon comprises a perimeter longitudinal length at a radial perimeter of the inverted neck balloon that is greater than an inner longitudinal length at a radially inwards position of the inverted neck balloon proximate the elongated member.

10. The catheter of claim 9, wherein the needle is configured to extend away from the central axis of the elongated member within at least a portion of the perimeter longitudinal length of the inverted neck balloon.

11. A chemical ablation system comprising:
the ablation catheter of claim 1; and
a medical device communicatively coupled to a lumen of the needle and configured to control delivery of a fluid via the lumen to the target treatment site.

12. A catheter comprising:
an elongated member configured to be navigated through vasculature of a patient to a target treatment site in a vessel of the patient;
a needle positioned at a distal portion of the elongated member, the needle configured to be within the elongated member when in a delivery configuration and radially extended to puncture a vessel wall of the vessel at the target treatment site when in a deployed configuration; and
an expandable inverted neck balloon, wherein when in an expanded configuration, a perimeter longitudinal length at a radial perimeter of the inverted neck balloon is greater than an inner longitudinal length at a radially inwards position of the inverted neck balloon proximate the elongated member,
wherein the expandable inverted neck balloon is configured to position a distal end of the needle at a predetermined distance from a central axis of the elongated member when the balloon is in the expanded configuration and the needle is in the deployed configuration.

13. The catheter of claim 12, wherein the expandable inverted neck balloon is attached to the elongated member at a first distal position proximal a distal end of the expandable inverted neck balloon, wherein the distal end of the expandable inverted neck balloon, when inflated, is distal to a distal end of the elongated member and radially outwards from the first distal position.

14. The catheter of claim 12, wherein the inverted neck balloon is configured to provide radial stabilization to the vessel wall proximate to the target treatment site and at a distal end of the expandable inverted neck balloon, wherein the needle is configured to puncture the vessel wall proximate the distal end of the expandable inverted neck balloon, and wherein the needle is configured to extend away from the central axis of the elongated member proximal to the distal end of the expandable inverted neck balloon and puncture the vessel wall distal to the distal end of the expandable inverted neck balloon.

15. The catheter of claim 12, wherein, when the balloon is in the expanded configuration, the balloon is configured maintain the elongated member substantially stationary relative to the vessel wall, wherein the balloon is configured to substantially radially center the elongated member within the vessel, and wherein the needle is configured to extend a predetermined radial distance into a target tissue at the target treatment site.

16. The catheter of claim 15, wherein, when the balloon is in the expanded configuration, the balloon is configured to apply a force to the vessel wall to urge a portion of the vessel contacted by the balloon to straighten, wherein, when the balloon is in the expanded configuration, the balloon is configured to reduce a curvature of the vessel wall proximate the target treatment site via repositioning at least a portion of the length of the vessel wall proximate the target treatment site.

17-19. (canceled)

20. The catheter of claim 1, wherein, when the balloon is in the expanded configuration, the balloon is configured to maintain the elongated member substantially stationary relative to the vessel wall.

21. The catheter of claim 1, wherein, when the balloon is in the expanded configuration, the balloon is configured to substantially radially center the elongated member within the vessel.

22. The catheter of claim 1, wherein, when the balloon is in the expanded configuration, the balloon is configured to reduce a curvature of the vessel wall proximate the target treatment site via repositioning at least a portion of the length of the vessel wall proximate the target treatment site.

23. A method comprising:
navigating a catheter through vasculature of a patient to a target treatment site in a vessel of the patient, wherein the catheter comprises:
an elongated member configured to be navigated through vasculature of a patient to the target treatment site;
a needle positioned at a distal portion of the elongated member, the needle configured to be within the elongated member when in a delivery configuration and radially extended to puncture a vessel wall of the vessel at the target treatment site when in a deployed configuration; and
an expandable balloon including a needle guide configured to position a distal end of the needle at a predetermined distance from a central axis of the elongated member when the balloon is in an expanded configuration and the needle is in the deployed configuration; and
expanding the balloon within the vasculature to maintain the elongated member substantially stationary relative to the vessel wall.

24. The method of claim 23, further comprising extending the needle a predetermined radial distance into a target tissue at the target treatment site, wherein the target tissue is a perivasculature adipose tissue proximate the vessel wall.

25. The method of claim 23, further comprising:
positioning, via expanding the balloon, a guide tube radially and circumferentially relative to the vessel wall within the vessel; and
stabilizing, via expanding the balloon, the guide tube from moving radially and circumferentially relative to the vessel wall, wherein the needle is configured to extend from the guide tube.
